(19) United States
(12) Patent Application Publication
(10) Pub. No.: US 2017/0164589 A1
(43) Pub. Date: Jun. 15, 2017

(54) MUTATION FOR TYPE I DIABETES MELLITUS AND ANIMAL MODEL

(71) Applicant: Medizinische Hochschule Hannover, Hannover (DE)

(72) Inventors: Sigurd LENZEN, Hannover (DE); Anne JOERNS, Hannover (DE); Dirk WEDEKIND, Neustadt am Rtibenberge (DE); Tanja ARNDT, Hannover (DE)

(21) Appl. No.: 15/308,969

(22) PCT Filed: May 5, 2015
(86) PCT No.: PCT/EP2015/059805

§ 371 Date: Nov. 4, 2016

(30) Foreign Application Priority Data
May 5, 2014 (EP) 14167082.8
May 9, 2014 (EP) 14167808.6

(51) Int. Cl. A01K 67/027; A61K 49/00

(52) U.S. Cl. CPC A01K 67/0278; A61K 49/0008; A01K 2267/0362; A01K 2227/10; A01K 2217/072

(57) ABSTRACT

The invention relates to a mutation in the Dock8 encoding gene that is causative for type 1 diabetes mellitus at least in the rat (rattus norvegicus) or mouse, the rat or mouse preferably having an MHC predisposing towards type 1 diabetes mellitus and/or a mutation in the von Willebrand factor Vwa2. Embodiments of the invention comprise a process for generating a non-human mammal by genetic manipulation to contain at least heterozygously, preferably homozygously, the mutation causative for type 1 diabetes mellitus, preferably in combination with an MHC predisposing for type 1 diabetes mellitus and/or with a mutation in the von Willebrand factor Vwa2.

The invention relates to a mutation that is causative for type 1 diabetes mellitus at least in the rat (rattus norvegicus) or mouse, the rat or mouse preferably having an MHC predisposing towards type 1 diabetes mellitus and/or a mutation in the von Willebrand factor Vwa2. Embodiments of the invention comprise a process for generating a non-human mammal by genetic manipulation to introduce in the non-human mammal at least heterozygously, preferably homozygously, the mutation causative for type 1 diabetes mellitus, preferably in combination with an MHC predisposing for type 1 diabetes mellitus and/or with a mutation in the von Willebrand factor Vwa2. Further, the invention relates to a non-human mammal that is genetically manipulated to contain at least heterozygously, preferably homozygously, the mutation causative for type 1 diabetes mellitus, preferably in combination with an MHC predisposing for type 1 diabetes mellitus and/or with a mutation in the von Willebrand factor Vwa2, a process for analysing the activity of a test compound by administration of the test compound to a non-human mammal containing at least heterozygously, preferably homozygously, the mutation causative for type 1 diabetes mellitus, preferably in combination with an MHC predisposing for type 1 diabetes mellitus and/or with a mutation in the von Willebrand factor Vwa2, and relates to a process for analysis of the genetic predisposition of a human being or of a non-human mammal for the genetic predisposition towards type 1 diabetes mellitus by analysing for the presence of the mutation, preferably in combination with analysing for the presence of an MHC predisposing towards type 1 diabetes mellitus and/or for a mutation in the von Willebrand factor Vwa2.

### STATE OF THE ART

Lenzen et al., Diabetologia 1189-1196 (2001), describe a spontaneous insulin-dependent diabetes mellitus rat strain designated LEW.1AR1/Ztm-iddm, having the MHC haplotype RT1.A^{a} B/D^{u} C^{u}. This rat develops diabetes at 58±2 d at 60% incidence without major sex preference, showing islets heavily infiltrated with B and T lymphocytes, macrophages and NK cells, destroying beta cells through apoptosis.

WO2005/054512 A2 describes the measurement of a type 1 diabetes mellitus specific gene product, as e.g. the expression of CDC42 among others was found elevated in type 1 diabetes mellitus patients.

Jörns et al., Diabetes 2041-2052 (2005), describe the immune cell infiltration comprising CD4+ T-cells, CD8+ T-cells and macrophages, and beta cell apoptosis during development of insulin-dependent diabetes mellitus in rat strain LEW.1AR1/Ztm-iddm.

Yang et al., Science 1398-1402 (2009) describe the activation of Rho GTPases by DOCK exchange factors and involvement of the DHR2 region of DOCK in Cdc42 binding.

### OBJECTIVE OF THE INVENTION

The objective of the invention is to provide a non-human experimental animal for use as an animal model for research purposes, especially for identification of compounds having activity against type 1 diabetes mellitus, as well as to provide a method for generating such a non-human experimental animal for research purposes. A further object is to provide an analytical process for analysing a human or a non-human mammal for its predisposition towards type 1 diabetes mellitus. A further objective is to provide a process for analysing the activity of a compound, especially against type 1 diabetes.

### DESCRIPTION OF THE INVENTION

The invention achieves the objectives by the features of the claims and specifically provides a mutant rat Dock8 encoding gene and mutant rat Dock8 protein and a mutant mouse Dock8 protein which in non-human mammals, preferably rodents, especially in rat (e.g. rattus norvegicus) and in mouse (mus musculus), induces insulin-dependent diabetes mellitus (iddm), closely resembling human type 1 diabetes. For the mutation, an autosomal recessive mode of inheritance with an incomplete penetrance of the diabetes phenotype of about 60% was found in the LEW.1AR1-iddm rat. The mutation was found in animals of the rat strain LEW.1AR1-iddm that showed type 1 diabetes (T1D), whereas the wild-type strain LEW.1AR1 is diabetes resistant and did not show this mutation. The mutant strain is designated LEW.1AR1-iddm. Both the wild-type strain LEW.1AR1 and the mutant strain LEW.1AR1-iddm carry the MHC II RT1-B/D^{u} haplotype which is indispensable for T1D in the rat.

Following linkage analysis on N2 backcross populations of the LEW.1AR1-iddm with the Brown Norway rat (BN) and respectively with the Paris rat (PAR), respectively, analyses of diabetic BN and PAR rats of N2 using SNP (single nucleotide polymorphisms) indicated candidate gene regions. Sequencing of candidate gene regions revealed a mutation in exon 44 of the dedicator of cytokinesis 8 gene (Dock8), resulting in an amino acid exchange from glutamine to glutamate at amino acid position 1864 (Q1864E) of Dock8 protein (SEQ ID NO: 1). The mutation is from the wild-type C to G at position 181980 of the Dock8 encoding sequence Dock8 (SEQ ID NO: 2), corresponding to position 228 622 763 on RNO1 (rattus norvegicus gene 1, RGSC genome assembly version 3.4). In SEQ ID NO: 2, which is based on RGSC genome assembly version 5.0, DHR2 is encoded by nucleotides No. 170118 to 192368, and exon 44 is at nucleotides No. 181971 to 182207.

The mutation resides in the β4 region of the DHR2 (DOCK homology region 2) of Dock8. The mutation is assumed to change the binding of the DHR2 region to Cdc42, resulting in the T1D phenotype.

The DHR2 region is at amino acids No. 1620-2070, preferably No. 1632-2070, exon 44 corresponds to amino acids No. 1861-1939, and the β4 region is at amino acids No. 1860-1867, preferably No. 1861-1867 of the rat Dock8 protein SEQ ID NO: 1 (mutated) and of SEQ ID NO: 10 (wild-type), respectively.

Accordingly, the invention in one embodiment relates to non-human mammals, e.g. rodents, preferably mouse and rat, which contain a mutation in the DHR2 encoding portion of the Dock8 encoding gene (Dock8), especially in the β4 region encoding gene region of DHR2, the mutation changing, e.g. increasing or decreasing, the interactions with Cdc42. Preferably the mutation causes the Q1864E mutation in the Dock8 (SEQ ID NO: 1) encoding region in the rat and the corresponding Q1865E mutation in the Dock8 (SEQ ID NO: 12) encoding region in the mouse.

In rat, the mutation Q1864E of Dock8 protein causes the infiltration of islets by T-cells with a predominance of CD8+ T-cells over CD4+ T-cells, whereas in the wild-type rat strain without this mutation, well-preserved beta cells were observed.

Further, the invention relates to a process for generating non-human mammals, e.g. rodents, preferably mouse and rat, by genetic manipulation to contain a mutation in the DHR2 encoding portion of the Dock8 encoding gene, especially in exon 44, preferably in the β4 region encoding gene of DHR2, the mutation changing, e.g. increasing or decreasing, the interactions with Cdc42. Preferably the non-human mammal is a rat or a mouse and the mutation causes the Q1864E mutation in Dock8 in the rat and respectively the Q1865E mutation in Dock8 in the mouse, respectively. In non-human mammals other than rat or mouse, the mutation preferably exchanges the amino acid Q for amino acid E in the site corresponding to position No. 1864 in rat Dock8 or corresponding to position No. 1865 in mouse Dock8, respectively. The amino acid corresponding to position 1864 in rat and 1865 in mouse can be found by sequence alignment, as Dock8 encoding genes have been found to be conserved, and preferably, the position is in the amino acids encoded by exon 44 and/or in the β4 region.

In a further embodiment, the invention relates to non-human mammals, e.g. rodents, preferably rat and mouse, having a gene encoding a mutation in the DHR2 encoding portion of the Dock8 encoding gene, especially in exon 44, preferably in the β4 region encoding gene of DHR2, the mutation changing, e.g. increasing or decreasing, the interactions with Cdc42. Preferably, the mutation causes the Q1864E mutation in Dock8 in the rat and the Q1865E mutation in Dock8 in the mouse, respectively, preferably homozygously. The non-human mammals can be generated by genetic manipulation introducing the mutation into the Dock8 encoding gene, e.g. by the process for generating non-human mammals by genetic manipulation or can be obtained by selection of mutant animals, e.g. of spontaneous or induced mutant mammals. Preferably these non-human mammals carry an MHC haplotype that is permissive for T1D, e.g. H²G⁷ haplotype in the mouse or RT1-B/D^{u} haplotype in the rat, optionally in combination with a mutation in the von Willebrandt factor Vwa2 encoding gene as described herein.

As the non-human mammals having such a mutation in the Dock8 encoding gene, preferably homozygously, are predisposed for T1D, the invention also relates to a process for the analysis of a compound with respect to its activity against T1D in the non-human mammal, e.g. a screening method. Generally, in the method, a compound to be tested is administered to the non-human mammal having such a mutation, preferably in combination with an MHC susceptible to T1D, e.g. H²G⁷ haplotype in the mouse or RT1-B/D^{u} haplotype in the rat, and/or in combination with a mutation in Vwa2, e.g. in exon 11 of the Vwa2 encoding gene, preferably causing the mutation R681W in rat Vwa2 or the mutation from R to W in a position corresponding to amino acid position No. 681 in rat Vwa2 protein (SEQ ID NO: 3), corresponding to amino acid position No. 159 in the amino acid sequence section encoded by exon 11 of the Vwa2 gene. The reaction of the non-human mammal to the compound administered is detected, e.g. by analysis of blood levels of glucose prior to and following administration of the compound.

The amino acid sequences of Dock8 are highly conserved between rat and mouse (98% amino acid identity), and between Dock8 in rat and human DOCK8 (92% amino acid identity).

Further, the invention relates to a nucleic acid sequence, optionally isolated, the nucleic acid sequence encoding non-human Dock8 or human DOCK8 encoding a mutation in the Dock8 and DOCK8, respectively, encoding gene causing a changed interaction of Dock8 and DOCK8, respectively, with Cdc42, or with human CDC42, respectively, which mutation is in the DHR2 encoding region, especially in exon 44, preferably in the 134 encoding section of the Dock8 and/or DOCK8 encoding gene, the mutation e.g. encoding a mutation of glutamine at an amino acid position corresponding to amino acid position 1864 (Q1864) in rat Dock 8 and human DOCK8, respectively, or at amino acid position 1865 (Q1865) in mouse Dock8, preferably to glutamate (E) resulting in the mutation Q1864E in rat Dock8 or human DOCK8 and Q1865E in mouse Dock8.

Further, the invention relates to an analytical process of determining in a sample obtained from a human or in a sample obtained from a non-human mammal mutations in the DHR2 encoding section, especially in exon 44, preferably in the β4 encoding section of the Dock8 encoding gene, the mutation e.g. encoding a mutation of glutamine at an amino acid position corresponding to amino acid position 1864 (Q1864) in rat or to amino acid position 1865 (Q1865) in mouse Dock8 (Dock8 mutant Q1865E in SEQ ID NO: 12, Dock8 wild-type in SEQ ID NO: 11) or to amino acid position 1864 in human DOCK8 (wild-type in SEQ ID NO: 8), preferably to glutamate (E) resulting in the mutation Q1864E in rat and respectively human, and Q1865E in mouse. The analytical process can e.g. comprise sequencing the DNA section encoding the non-human Dock8 or the human DOCK8, including the section encoding the amino acids of DHR2, preferably of the β4 region encoding section, preferably exon 44.

It has further been found that the diabetes-prone rat strain LEW.1AR1-iddm carries an SNP (single nucleotide polymorphism) in exon 11 (amino acids No. 523 . . . 707 in SEQ ID NO: 3) of the von Willebrand factor Vwa2 encoding gene, at position 263216228 of the RGSC Genome Assembly, version 3.4 of RNO, resulting in a change of the amino acid sequence (SEQ ID NO: 3) of Vwa2 from the wild-type arginine (R) to tryptophan (W) at amino acid position 681 (R681W).

Accordingly, in a preferred embodiment, in the processes and non-human mammals according to the invention, the Vwa2 encoding gene has a sequence causing a mutation R681W in the Vwa2 encoding gene, corresponding to amino acid position 159 of the amino acids encoded by exon 11 of the Vwa2 encoding gene.

The invention is now described in greater detail with reference to the figures, which show in

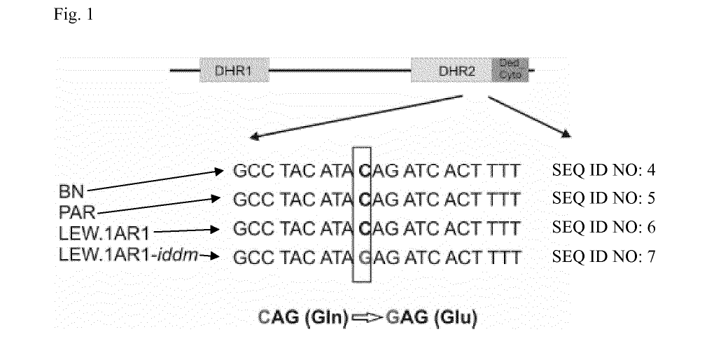

FIG. 1 a schematic drawing of rat Dock8 including the mutation in exon 44,

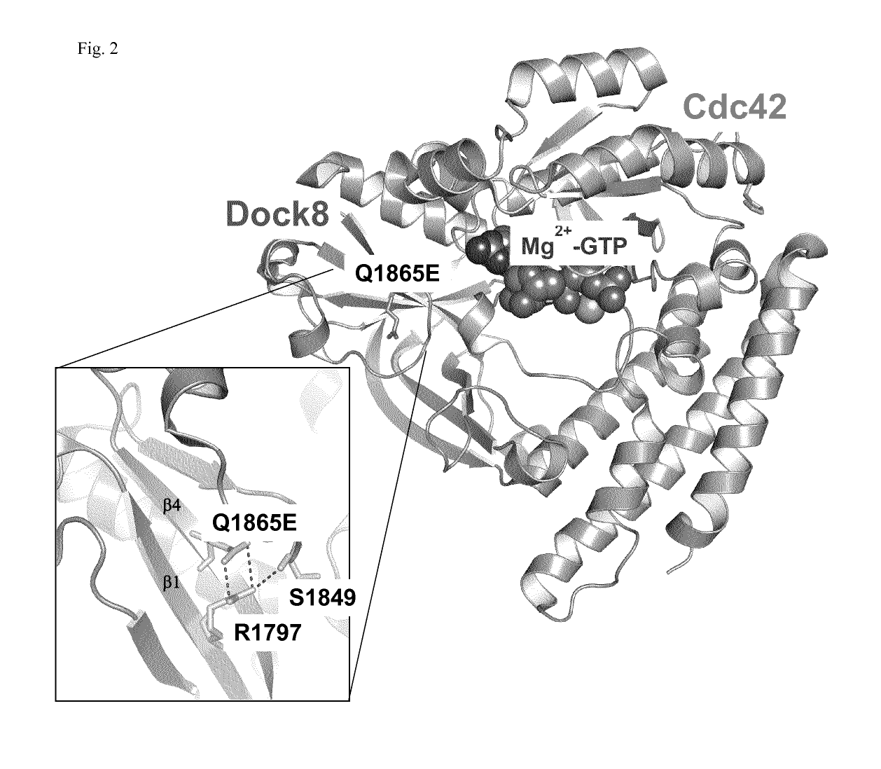

FIG. 2 a graphic model of the DHR2 region of mouse Dock8 in the complex with Cdc42,

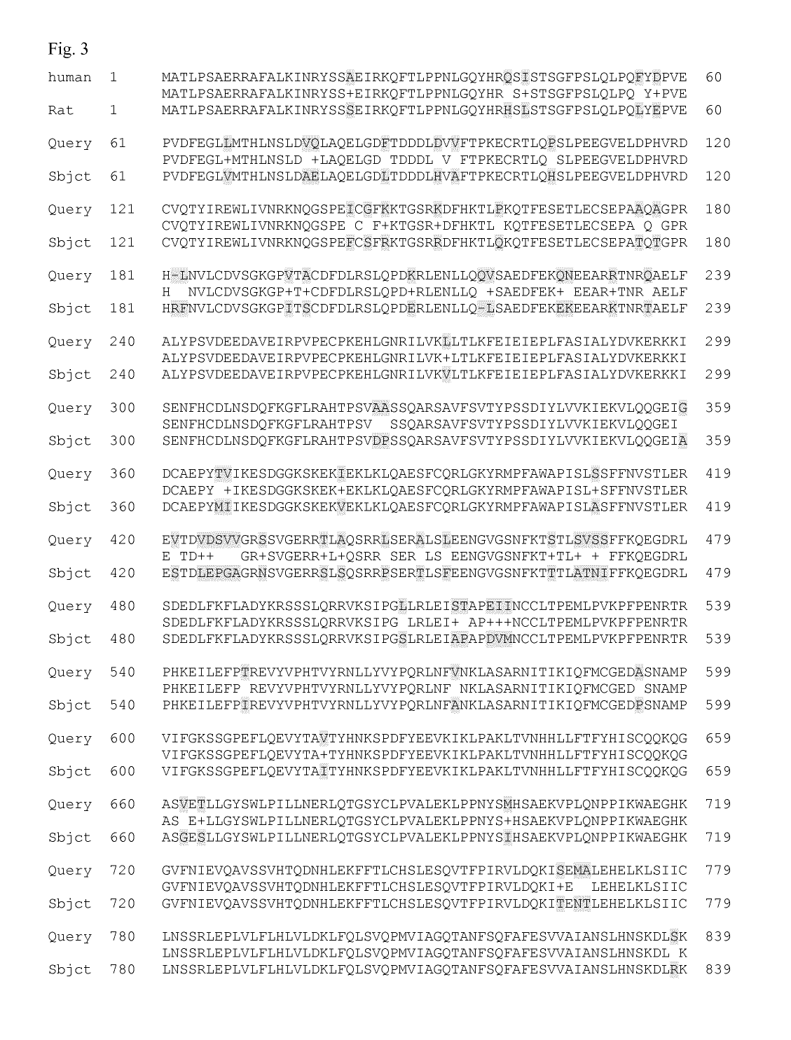

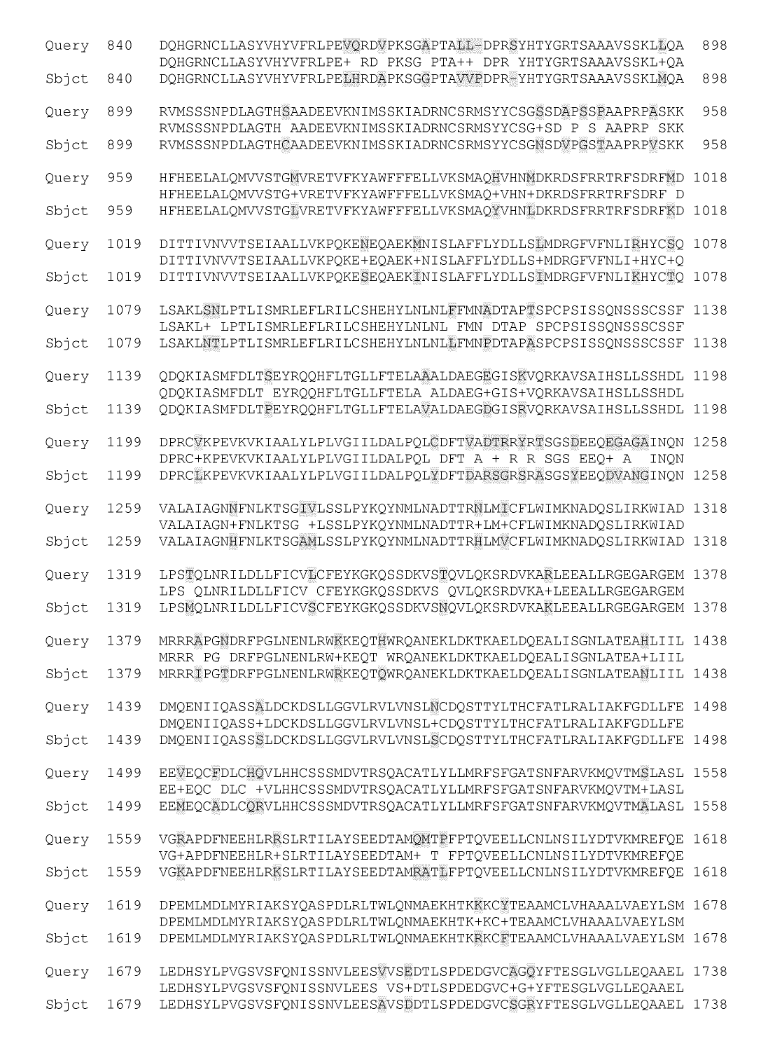

FIG. 3 a sequence alignment of amino acid sequences of DOCK8 of human origin (Query) and of rat origin (Sbjct), both wild-type, with the centre line showing the consensus,

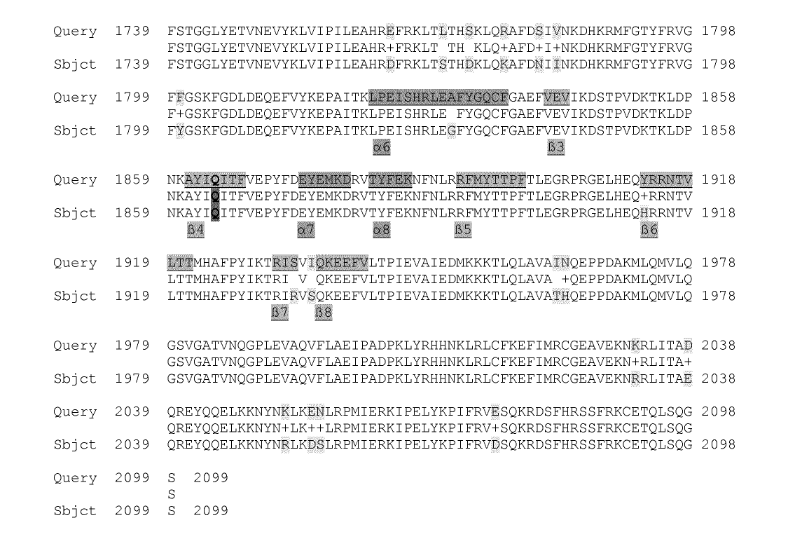

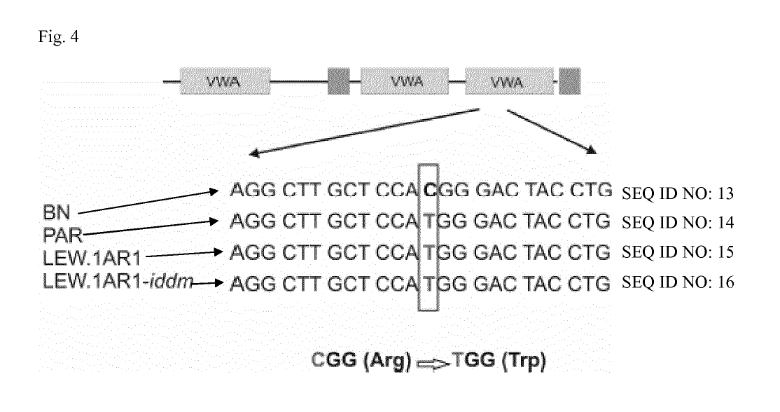

FIG. 4 a schematic drawing of the structure of the gene encoding Vwa2 and the mutation in exon 11,

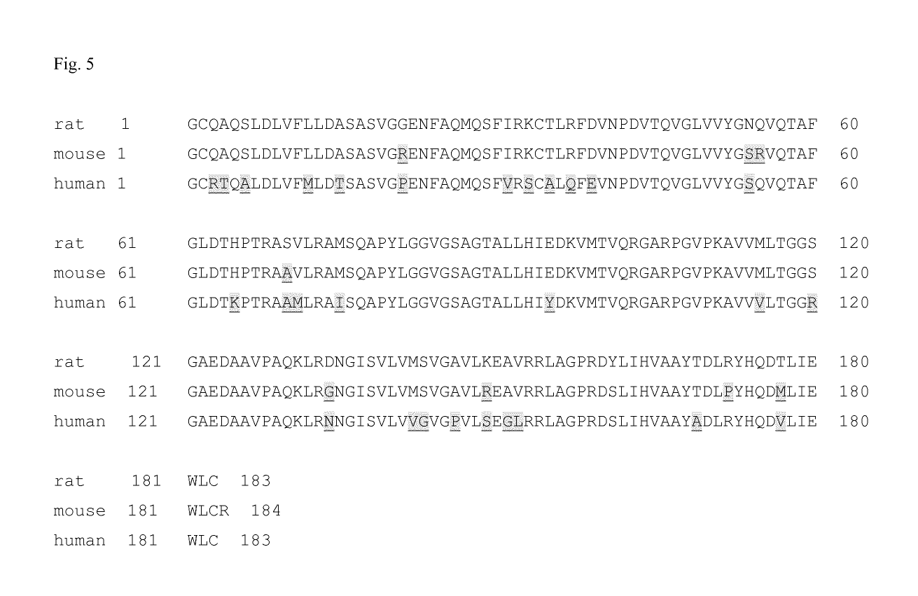

FIG. 5 a sequence alignment of amino acid sequences of exon 11 of Vwa2 of rat origin, of mouse origin and of human origin, wild-type each,

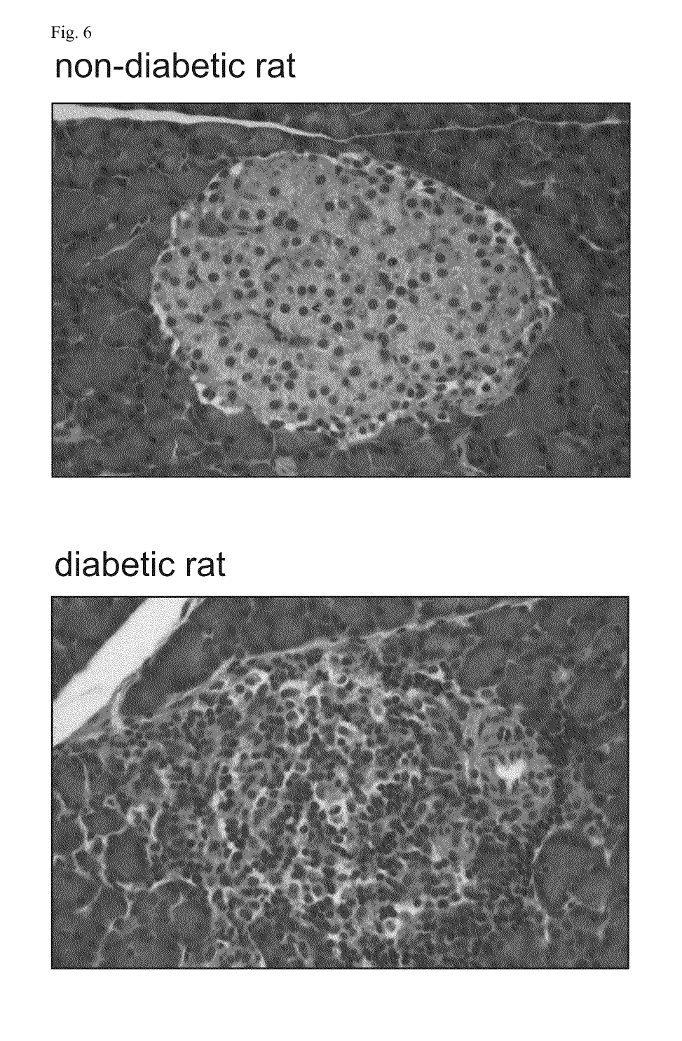

FIG. 6 micrographs of immune cells of pancreas sections from rat with HE staining, and in

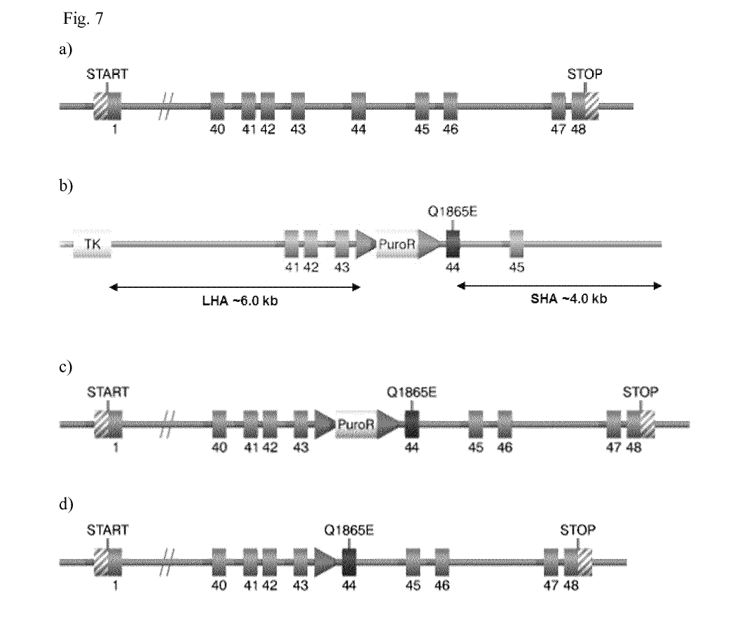

FIG. 7 schematically the cloning strategy in the process of generating a transgenic mouse according to the invention.

A section of the structure of the gene encoding rat Dock8 is schematically shown in FIG. 1. The gene contains 48 exons, and the mutation that causes a change in the binding of Dock8 to Cdc42 is located in exon 44. For comparison, a section from the nucleotide sequence of exon 44 of the Brown Norway rat (BN, SEQ ID NO: 4), the Paris rat (PAR, SEQ ID NO: 5), the wild-type strain LEW.1AR1 (SEQ ID NO: 6), and the mutant strain LEW.1AR1-iddm (SEQ ID NO: 7) are aligned. This sectional alignment shows that only the T1D phenotypic rat of the invention carries the mutation of C to G, resulting in the change from encoding glutamine (Gln) to glutamate (Glu) at amino acid position 1864 of Dock8 in human (DOCK8) and rat, and respectively at corresponding amino acid position 1865 in mouse Dock8.

FIG. 2 shows a graphic model of the mouse Q1865E mutated DHR2 region of Dock8 in complex with Cdc42, with Mg²⁺ and GTP bound. The graphic model was prepared using the PyMOL Molecular Graphics System, version 1.5.0.4, Schrodinger LLC using the PDB-code 3VHL. Due to the high homology of the mouse Dock8 protein to the rat Dock8 protein and the mouse Dock8 carrying the mutation encoding Q1865E and the mouse carrying the mutation encoding Q1864E, the graphic model of the mouse Dock8 was used.

The glutamate residue at position 1865 (Q1865E) of mouse Dock8 is shown as a stick model within the β4 section of the DHR2 region. The enlarged inset shows that according to this model, the mutation at position 1865 to glutamate can result in formation of a salt bridge with the adjacent arginine in position 1797 (R1797), located in β1, and to interact with the serine at position 1849 (S1849), which is part of a flexible loop emerging from the α6 helix. The loop of the α6 helix is critically involved in the interaction with Cdc42, which interaction can be influenced by the stronger interaction of the glutamate residue at position 1865.

FIG. 3 shows a sequence alignment between the wild-type amino acid sequences of Dock8 from rat (lower row, SEQ ID NO: 10) and human origin (upper row, SEQ ID NO: 8) with the consensus sequence (centre row, SEQ ID NO: 9). Differences in amino acids are marked by shading individual amino acids. The wild-type Q in position 1864 is also marked. The high homology between Dock8 of rat origin and DOCK8 of human origin as well as of mouse origin shows that a non-human mammal carrying the mutation in the DHR2 region, exemplified by the Q1864E mutation in the rat, is a valuable laboratory model representing human T1D. Further, the high homology shows that an analytical process for determining mutations in the DHR2 region in human beings, which mutations affect the interaction of the DHR2 region with Cdc42, especially mutations in the β4 region, e.g. in exon 44, e.g. a Q1864E mutation, is suitable for identifying the genetic predisposition towards T1D, especially combination with MHC RT1-B/D^{u} in the rat, MHC H²G⁷ haplotype in the mouse, and MHC HLA-DR3 or HLA-DR4 in a human respectively, and/or each in combination with a mutation in the von Willebrand factor Vwa2, e.g. in its exon 11, exemplified by the R681W mutation, e.g. in the rat SEQ ID NO: 3. In FIG. 3, α-helix and β-folding sheet regions are indicated below the amino acids, and amino acids forming the a-helix and β-folding sheet regions are underlined.

The amino acid sequence of mouse Dock8 wild-type is given as SEQ ID NO: 11, the mouse Q1865E mutant Dock8 as SEQ ID NO: 12. In SEQ ID NO: 11 and SEQ ID NO: 12, respectively, the DHR2 region is at amino acids No. 1633-2071, amino acids encoded or corresponding to exon 44 are at No. 1862-1940, and the β4 region is at amino acids No. 1862-1868.

FIG. 4 shows the structure of the gene encoding Vwa2, including the T of the SNP in exon (ex) 11, resulting in a change of arginine (Arg, R) to tryptophan (Trp, W) at amino acid position 681 (R681W). The amino acid exchange is located in the third Vwa domain of Vwa2. The sectional sequence comparison shows that this mutation is not found in the Brown Norway (BN) but also in the Paris (PAR) and the LEW.1AR1 rats as well as in the mutant LEW.1AR1-iddm rat.

This mutation of Vwa2 is assumed to contribute to a diabetic T1D phenotype in combination with the mutation of Dock8.

Accordingly, a mutation in the Vwa2 encoding gene, especially in its third VWA domain, preferably resulting in a tryptophan in amino acid position 681, is preferred in the non-human mammals of the invention and in the processes of the invention for generating non-human mammals and for analysing a sample of mammalian origin, including human origin, in respect of predisposition towards T1D.

FIG. 5 shows an amino acid alignment of the wild-type Vwa2 amino acids encoded by exon 11 of Vwa2 of rat (SEQ ID NO: 17), of mouse (SEQ ID NO: 18), and of human origin (SEQ ID NO: 19), wild-type each. Amino acids encoded by exon 11 of Vwa2 that differ in mouse or human when compared to the amino acid sequence encoded by rat exon 11 of the Vwa2 gene are indicated by underlining The high degree of sequence identity and especially of the amino acid R in position 159 for each Vwa2 exon 11, corresponding to position 681 in mutant Vwa2 of rat in SEQ ID NO: 3 shows that a Vwa2 having a mutation in amino acid position 159 of exon 11 of Vwa2 also in mouse causes a genetic predisposition for T1D. Accordingly, the process for producing a non-human mammal preferably comprises introducing a mutation in amino acid position 159 in the amino acid sequence encoded by exon 11 of the Vwa2 encoding gene, especially a mutation R159W, and the non-human mammal preferably contains a mutation in amino acid position 159 in the amino acid sequence encoded by exon 11 of Vwa2, especially a mutation R159W in the amino acid sequence encoded by exon 11 of the Vwa2 encoding gene. In rat, the exon 11 of the Vwa2 encoding gene preferably encodes SEQ ID NO: 17 in exon 11, in mouse preferably SEQ ID NO: 18 in exon 11, each with the mutation R159W. The wild-type amino acid sequences of Vwa2 of rat is given as SEQ ID NO: 20 (exon 11 encoding amino acids No. 522-706), of mouse as SEQ ID NO: 21 (exon 11 encoding amino acids No. 522-706), and of human VWA2 as SEQ ID NO: 22 (exon 11 encoding amino acids No. 523-707). In the rat and mouse Vwa2, amino acid position 159 of exon 11 corresponds to amino acid position 681 in SEQ ID NO: 20 and SEQ ID NO: 21, respectively, and to amino acid position 682 in SEQ ID NO: 22 in human VWA2.

Accordingly, the analytical process for the genetic predisposition for T1D, both in a human sample and in a non-human mammalian sample, preferably comprises determining a mutation in amino acid position 159 encoded by exon 11 of Vwa2, especially determining a mutation R159W in the amino acid sequence encoded by exon 11 of Vwa2.

FIG. 6 shows micrographs of pancreatic islets with beta cells with haematoxylin (HE) staining from a non-diabetic LEW.1AR1 rat and of a diabetic rat, exemplified by LEW.1AR1-iddm. The micrographs show that the only known genetic difference between these rats, namely the Q1864E mutation of SEQ ID NO: 1, results in the disturbed morphology of the islets in the diabetic rat and in apoptosis of beta cells.

Accordingly, it is preferred, that the mutation of the DHR2 region of Dock8 causing a changed interaction with Cdc42 in the non-human mammal is homozygous. Further, it is preferred in the analytical process that in the non-human mammal and in the human, respectively, the haplotype is determined in respect of mutations in the Dock8 encoding gene.

The DNA sequence of the wild-type mouse Dock8 encoding gene is given at SEQ ID NO: 23, based on the mouse chromosome 19 (MMU19, GRCm38 genome assembly), positions 24,999,529-25,202,432, containing the DHR2 region at nucleotides No. 177307-200987, exon 44 at nucleotides No. 188908-189045, and the wild-type C at position No. 188818, which according to the invention is mutated to G to encode the amino acid exchange Q1865E in Dock8 protein.

Further analysis of pancreatic islets with beta cells from diabetic animals, exemplified by LEW.1AR1-iddm rats in comparison to those form non-diabetic LEW.1AR1-iddm rats by DAB immunohistochemistry with haematoxylin counterstaining with immunostaining for CD4+ (CD4+ T-cells), for CD8+ (CD8+ T-cells), for insulin, and for CD68+(macrophages) shows that CD8+ T-cells and CD68+ macrophages with a smaller number of CD4+ T-cells infiltrate pancreatic islets as well as a severely reduced insulin production in the diabetic rats. The direct comparison between diabetic LEW.1AR1-iddm rats carrying the Q1864E mutation of Dock8 protein and non-diabetic LEW.1AR1 rats which otherwise have the same genetic background shows that a mutation within the DHR2 region of Dock8 protein to change the interaction with Cdc42 causes T1D, at least in the genetic background of MHC II RT1-B/D^{u} haplotype, preferably additionally with a mutation in the von Willebrandt factor Vwa2, especially in exon 11 of Vwa2, encoding e.g. R681W in the Vwa2 protein (SEQ ID NOs: 20, 21, 22), corresponding to R159W in the amino acid sequence section encoded by exon 11 (SEQ ID NOs: 17, 18, 19).

### EXAMPLE

### Transgenic Non-Human Mammal

A transgenic mouse carrying the Q1865E mutant DOCK8 encoding gene can be generated using targeted mutagenesis in mouse embryonic stem (ES) cells. ES cells of the mouse strain C57B1/6 N Tac were transfected using electroporation with a linearized nucleic acid vector having homologous sections at both the 5′-end and the 3′-end for recombination within the DOCK8 encoding gene, a resistance gene (Puromycin) flanked by frt sites for later removal of the resistance gene. A schematic representation of the mouse genomic locus is shown in FIG. 7a), and of the vector in FIG. 7b), wherein TK designates the selection marker thymidine kinase, LHA designates the 5′ long homology arm, SHA designates the 3′ short homology arm, and numbers indicate exons. The mutation Q1865E to be introduced in exon 44 is contained in the vector. FIG. 7c) shows the integrated vector. The Puromycin resistance gene (PuroR) is flanked by FRT sites is located in intron 43 and allows for in vivo Flp-mediated removal of the selection marker following selection, as shown in FIG. 7d).

Transfected cells were selected for the resistance carried by the vector and insertion of the vector at the target site was controlled by Southern blotting after restriction of total DNA or by PCR on total DNA spanning the insertion site. Homologous recombinant ES cells were grown on a mitotically inactivated feeder layer of mouse embryonic fibroblasts in ES cell culture medium containing leukemia inhibitory factor and fetal bovine serum for expansion and frozen in liquid nitrogen. Subsequently, the transgenic cells are injected into blastocysts and implanted into a foster mouse. Chimeric offspring are crossed, and the mice that are homozygous for the mutation encoding Dock8 having SEQ ID NO: 12 with the mutation Q1865E are selected.

1. Process for generating a non-human mammal having a genetic predisposition towards type 1 diabetes mellitus, characterized by introducing into the mammal a mutation within the Dock homology region-2 (DHR2) region of the dedicator of cytokinesis 8 (Dock8) encoding gene, the mutation encoding an amino acid altering the binding of the DHR2 region of Dock8 to Cdc42.

2. Process according to claim 1, characterized in that the mutation is within the exon 44, preferably in the section encoding the β4 region of the DHR2 region.

3. Process according claim 1, characterized in that the mutation encodes an amino acid exchange in the Dock8 encoding gene corresponding to Q1864E of SEQ ID NO: 1 or Q1865E of SEQ ID NO: 12.

4. Process according to claim 1, characterized in that the mammal is a mouse having the MHC haplotype H²G⁷ and/or carrying a mutation in the von Willebrand factor A2 (Vwa2) encoding gene encoding an amino acid exchange in amino acid position 159 of the amino acid sequence encoded by exon 11 of the Vwa2 (SEQ ID NO: 17 or SEQ ID NO: 18) encoding gene.

5. Polynucleic acid molecule comprising the section of the gene encoding the DHR2 region of the Dock8 protein, characterized by comprising a mutation within the section encoding the DHR2 region, the mutation encoding an amino acid alteration reducing binding of the DHR2 region of Dock8 to Cdc42.

6. Polynucleic acid molecule according to claim 5, characterized in that the mutation is within the exon 44, preferably in the section encoding the 04 region of the DHR2 region.

7. Polynucleic acid molecule according to claim 5, characterized in that the mutation encodes the amino acid exchange Q1864E in a Dock8 protein according to SEQ ID NO: 1, or the mutation encodes the amino acid exchange Q1865E in a Dock8 protein according to SEQ ID NO: 12.

8. Non-human mammal having a genetic mutation predisposing for type 1 diabetes, characterized by having a mutation in the section encoding the DHR2 region of the Dock8 protein encoding gene, the mutation encoding an amino acid alteration reducing binding of the DHR2 region of Dock8 to Cdc42.

9. Non-human mammal according to claim 8, characterized in that the mutation is within the exon 44, preferably in the β4 section of the DHR2 region.

10. Non-human mammal according to claim 8, characterized in that the mammal is a rat and the mutation encodes the amino acid exchange Q1864E in a Dock8 protein according to SEQ ID NO: 1, or that the mammal is a mouse and the mutation encodes the amino acid exchange Q1865E in a Dock8 protein according to SEQ ID NO: 12.

11. Non-human mammal according to claim 8, characterized in that the mammal is a rat and has a mutation encoding a Willebrand factor A2 (Vwa2) protein having an amino acid in amino acid position 159 of exon 11 (SEQ ID NO: 17 or SEQ ID NO: 18) other than arginine (R).

12. Process for analysis of the activity of a compound by administration of the compound to a non-human mammal, characterized in that the non-human mammal is according to claim 8 which is homozygous for a mutation in the section encoding the DHR2 region of the Dock8 protein encoding gene.

13. Process according to claim 12, characterized in that the mammal is a mouse having the MHC haplotype H²G⁷ or the mammal is a rat having the MHC haplotype RT1-B/D^{u}.

14. Process for analysis of a sample obtained from a human or from a non-human mammal for genetic predisposition towards type 1 diabetes mellitus, characterized by determining the presence of mutations within the DHR2 region of the DOCK8 encoding gene, the mutation encoding an amino acid alteration reducing binding of the DHR2 region of DOCK8 to Cdc42.

15. Process according to claim 14, characterized in that the mutation encodes an amino acid at position 1864 in the rat gene encoding Dock8 of SEQ ID NO: 10 or in the human gene encoding DOCK8 of SEQ ID NO: 8 or the mutation encodes an amino acid at position 1865 in the mouse gene encoding Dock8 of SEQ ID NO: 11, and/or comprising determining the presence of a mutation encoding an amino acid at position 681 in the rat gene encoding the von Willebrand factor A2 (Vwa2) of SEQ ID NO: 20 or in the mouse gene encoding the von Willebrand factor A2 (Vwa2) of SEQ ID NO: 21 or the mutation encodes an amino acid at position 682 of the human gene encoding the von Willebrand factor A2 (Vwa2) of SEQ ID NO: 22.
